(19) United States
(12) Patent Application Publication
(10) Pub. No.: US 2025/0334522 A1
(43) Pub. Date: Oct. 30, 2025

(54) FEMTO-LEVEL DETECTION OF ANALYTE USING SURFACE ENHANCED RAMAN SCATTERING (SERS) SUBSTRATE AND PROCESS THEREOF

(71) Applicant: KING FAHD UNIVERSITY OF PETROLEUM AND MINERALS, Dhahran (SA)

(72) Inventors: Nasurullah Abdul Wahid MAHAR, Dhahran (SA); Abdulaziz Abdulrahman AL-SAADI, Dhahran (SA)

(73) Assignee: KING FAHD UNIVERSITY OF PETROLEUM AND MINERALS, Dhahran (SA)

(21) Appl. No.: 18/650,409

(22) Filed: Apr. 30, 2024

(51) Int. Cl. G01N 21/65; G01N 33/49

(52) U.S. Cl. CPC G01N 21/658; G01N 33/49; G01N 2201/0612; G01N 2201/0621

(57) ABSTRACT

A surface-enhanced Raman scattering (SERS) substrate and system for detecting benzamide compounds of Formula (I). The SERS substrate includes a transparent substrate; and a layer of triangle-shaped silver nanoprisms (AgNPMs) at least partially covering a surface of the transparent substrate. The SERS substrate can detect a benzamide compound with a detection of from 1×10⁻¹⁴ to 1×10⁻¹⁰ molar (M). A method of obtaining the Raman spectrum of an analyte in a solution using the SERS substrate.

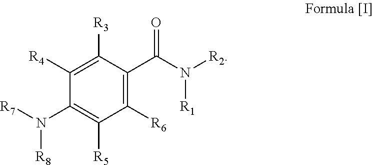

### STATEMENT OF PRIOR DISCLOSURE BY AN INVENTOR

Aspects of the present disclosed are described in Nasurullah Mahar and Abdulaziz A. Al-Saadi, “Light-induced synthesis of silver nanoprisms as a surface-enhanced Raman scattering substrate for N-acetyl procainamide drug quantification” Spectrochimica Acta Part A: Molecular and Biomolecular Spectroscopy 302 (2023) 122996 which is incorporated by reference in its entirety.

### STATEMENT OF ACKNOWLEDGEMENT

Support provided by King Fahd University of Petroleum & Minerals (KFUPM) through project no. DF191043 is gratefully acknowledged.

### BACKGROUND

### Technical Field

The present disclosure is related to the detection of analytes up to femto level concentrations and substrates used in their detection using techniques such as Surface-Enhanced Raman scattering (DSERS) and methods of preparing SERS substrates.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

A non-invasive method for detecting analytes in blood would be beneficial for determining analyte concentration without painful intervention and/or the use of any reagents in medical diagnostics. Surface-enhanced Raman spectroscopy (SERS) is one such technique. SERS is a powerful vibrational spectroscopy that allows for highly sensitive structural detection of low-concentration analytes by the amplification of electromagnetic fields generated by the excitation of localized surface plasmons [See: Sharma et al. SERS: Materials, applications, and the future, Materials Today 15(2012) 16-25]. The proximity of a Raman active molecule adsorbed on a metal surface can enhance the signal of the Raman active molecule, and the intensity arising from the Raman signal. This enhancement is the SERS effect. SERS can be used with adsorbates on a limited number of metal surfaces having a precisely prepared roughened surface. However, the large surface enhancement coupled with the need for a specific molecule to be adsorbed on the surface makes the technique prone to interference [Markovic et al. Surface-Enhanced Raman Scattering (SERS) Biochemical Applications, Encyclopedia of Spectroscopy and Spectrometry (Third Edition), 2017]. In the last 30 years, researchers strived to optimize substrate structure and maximize enhancement factors [See; Sharma et al. SERS: Materials, applications, and the future, Materials Today 15 (2012) 16-25]. There have been reports, CN115818554A, on the use of silver nanosphere-based SERS for detection at a single molecule level. CN117120828A involves a plurality of capture molecules directly bound to the Raman-active linker molecule that produce enhanced Raman signals. In another report, a highly sensitive approach based on SERS spectroscopy was developed for the detection of the procainamide drug using gold nanoparticles for the enhancement of Raman intensities [See: Mahar et al. Spectroanalytical SERS-based detection of trace-level procainamide using green-synthesized gold nanoparticles, Surfaces and Interfaces 31(2022) 102059]. However, there is a continuous demand and need to achieve selectivity and sensitivity towards an analyte in biological fluids, considering the plethora of biomolecules present. Though SERS have an enhanced signal, achieving the desired sensitivity and selectivity by using a substrate will remain a continuous exploration.

Accordingly it is one object of the present disclosure to provide a SERS substrate and method for detecting analytes as low concentrations, especially benzamide compounds at femto level concentrations.

### SUMMARY

In an exemplary embodiment, a surface-enhanced Raman scattering (SERS) substrate. The SERS comprising a transparent substrate, a layer of triangle-shaped silver nanoprisms (AgNPMs) at least partially covering a surface of the transparent substrate. The SERS substrate can detect a benzamide compound with a detection of from 1×10⁻¹⁴ to 1×10⁻¹⁰ molar (M).

In some embodiments, the transparent substrate comprises a glass substrate.

In some embodiments, the triangle-shaped silver nanoprisms (AgNPMs) have an average particle size of from 70 to 120 nanometers (nm). In some embodiments, the triangle-shaped silver nanoprisms have an average particle size of about 95 nm.

In some embodiments, the benzamine compound has a formula (I)

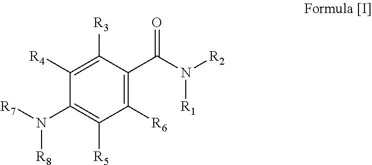

wherein R₁, R₂, R₇, and R₈ are each independently selected from the group consisting of a hydrogen atom, an optionally substituted alkyl, an optionally substituted aryl, and an optionally substituted heterocyclic aryl; and wherein R₃, R₄, R₅, and R₆ are each independently selected from the group consisting of a hydrogen atom, a halogen atom, an optionally substituted alkyl.

In some embodiments, the benzamide compound is N-acetyl procainamide (NAPA), wherein the SERS substrate has a detection limit of 0.5×10⁻¹³ M.

In an exemplary embodiment, a method of forming the SERS substrate is described. The method involves preparing the triangle-shaped silver nanoprisms by mixing silver nanospheres and a phosphine ligand in an alkaline solution to form a mixture, aging the mixture in a dark environment, and exposing the mixture to light, thereby converting silver nanospheres to the triangle-shaped silver nanoprisms.

In some embodiments, the silver nanospheres have an average particle size in a range of 20 to 120 nm.

In some embodiments, the phosphine ligand is at least one of a bis (p-sulfonatophenyl) phenylphosphine dihydrate dipotassium (BSPP) salt, and a triphenylphosphine-3,3′,3″-trisulfonic acid trisodium salt.

In some embodiments, the alkaline solution comprises at least one of LiOH, NaOH, KOH, and Ca(OH)₂.

In some embodiments, a molar ratio of the silver nanospheres to the phosphine ligand is in a range of 2:1 to 1:2.

In some embodiments, the light source is a monochromatic light emitting diode (LED) light having a wavelength of from 400 to 500 nm under a powder of from 120 to 180 watts (W).

In some embodiments, the monochromatic LED light has a wavelength of about 455 nm under a powder of about 150 W.

In an exemplary embodiment, a method of obtaining a Raman spectrum of an analyte in a solution is described. The method comprises contacting the solution with SERS substrate to form a sample, exposing the sample to Raman laser light such that a portion of the Raman laser light is scattered by the sample to form scattered light, and detecting the scattered light, wherein the analyte is N-acetyl procainamide (NAPA).

In some embodiments, the solution is human blood.

In some embodiments, the scattered light is monitored from 400-2,000 cm⁻¹.

In some embodiments, the method includes quantifying the amount of NAPA present in the solution based on the intensity of the scattered light.

In some embodiments, the intensity of the scattered light linearly correlates with the amount of NAPA present in the solution.

In some embodiments, a linear dynamic range of NAPA present in the solution is from 0.5×10⁻¹² to 0.5×10⁻⁴ M.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a flow chart depicting a process of preparing triangle-shaped silver nanoprisms (Ag-NPM), according to certain embodiments.

FIG. 1B is a flow chart depicting a method of obtaining a Raman spectrum of an analyte, according to certain embodiments.

FIG. 2 is a schematic illustration depicting photo-induced synthesis of Ag-NPM from silver nanospheres (Ag-NS) and their respective roles as efficient SERS substrates, according to certain embodiments.

FIG. 3 shows ultraviolet-visible (UV-Vis) spectra of the Nanoprisms for the time period of 6 weeks, according to certain embodiments.

FIG. 4 shows ultraviolet-visible-near infrared (UV-Vis-NIR) spectra depicting the formation of silver nanoprisms (AgNPMs) from silver nanospheres (AgNSs), according to certain embodiments.

FIG. 5A shows the field emission scanning electron microscope (FE-SEM) image of AgNSs.

FIG. 5B shows the FE-SEM image of the AgNPMs showing mixed morphologies, according to certain embodiments.

FIG. 5C is a histogram comparing the average particle sizes of the AgNSs, according to certain embodiments.

FIG. 5D is the average particle size of the AgNPMs, according to certain embodiments.

FIG. 6A shows UV-Vis spectra of the free N-acetyl procainamide (NAPA) drug, according to certain embodiments.

FIG. 6B shows excitation UV-vis-NIR spectra of the photo-induced AgNPMs after interacting with the NAPA drug, according to certain embodiments.

FIG. 7 shows Raman spectra of AgNSs and of AgNPMs, acquired at 633 nm excitation source, using 50× lens, 1800 grating, 20 sec. acquisition time and 4-sec. accumulation, according to certain embodiments.

FIG. 8 shows the SERS spectra of NAPA alone and in combination with AgNSs and AgNPMs, acquired at a 633 nm excitation source using a 50× lens, 20 sec. acquisition time, and 4-sec. accumulation, according to certain embodiments.

FIG. 9A shows the peak intensity-concentration dependence of the NAPA molecules in contact with the AgNPMs substrate, according to certain embodiments.

FIG. 9B shows the peak intensity-concentration dependence of the NAPA molecules in contact with the AgNSs substrate, according to certain embodiments.

FIG. 9C compares the near dynamic ranges and correlation coefficients of the intensity concentration relationship for AgNPMs and AgNSs, according to certain embodiments.

FIG. 10A shows interference effect of different interferents, namely sulindac (SD), ciprofloxacin (CPS), citric acid (CA), and valeric acid (VA), 10⁻² M each, mixed with NAPA at 10⁻⁶ M concertation, according to certain embodiments.

FIG. 10B shows selectivity outcome in terms of SERS intensity (1652 cm⁻¹) of the probe molecules (10⁻⁶ M) in presence of the interferents, according to certain embodiments.

FIG. 10C shows stability of the NPMs-NAPA solution spectra taken over a time period of 30 days, according to certain embodiments.

FIG. 11 shows the SERS spectral stability of AgNPMs-NAPA solution spectra taken over a 30-day period, according to certain embodiments.

### DETAILED DESCRIPTION

In the drawings, reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values there between.

As used herein, “SERS” refers to surface-enhanced Raman scattering, and SERS substrate refers to a substrate used for detection of an analyte.

As used herein, the enhancement factor (EF) referred to herein is calculated based on the equation (1) given in this invention.

As used herein, the term “alkyl” unless otherwise specified refers to both branched and straight chain aliphatic (non-aromatic) hydrocarbons which may be primary, secondary, and/or tertiary hydrocarbons typically having 1 to 32 carbon atoms (e.g., C₁, C₂, C₃, C₄, C₅, C₆, C₇, C₈, C₉, C₁₀, C₁₁, C₁₂, C₁₃, C₁₄, etc.) and specifically includes, but is not limited to, saturated alkyl groups such as methyl, ethyl, propyl, isopropyl, butyl, isobutyl, t-butyl, pentyl, isopentyl, neopentyl, hexyl, isohexyl, 3-methylpentyl, 2,2-dimethylbutyl, 2,3-dimethylbutyl, 2-ethylhexyl, heptyl, octyl, nonyl, 3,7-dimethyloctyl, decyl, undecyl, dodecyl, tridecyl, tetradecyl, pentadecyl, hexadecyl, heptadecyl, octadecyl, nonadecyl, eicosyl, guerbet-type alkyl groups (e.g., 2-methylpentyl, 2-ethylhexyl, 2-proylheptyl, 2-butyloctyl, 2-pentylnonyl, 2-hexyldecyl, 2-heptylundecyl, 2-octyldodecyl, 2-nonyltridecyl, 2-decyltetradecyl, and 2-undecylpentadecyl), as well as unsaturated alkenyl and alkynyl variants such as vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, oleyl, linoleyl, and the like.

The term “aryl” means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, anthracenyl, indanyl, 1-naphthyl, 2-naphthyl, and tetrahydronaphthyl. The fused aryls may be connected to another group either at a suitable position on the cycloalkyl/cycloalkenyl ring or the aromatic ring.

As used herein, the term “heteroaryl or heterocyclic aryl” is intended to mean stable monocyclic and polycyclic aromatic hydrocarbons that include at least one heteroatom ring member such as sulfur, oxygen, or nitrogen. Heteroaryl groups are heterocyclyl groups which are aromatic, and may include, without limitation, pyridyl, pyrrolyl, pyrimidinyl, pyrazinyl, pyridazinyl, triazinyl, furyl, quinolyl, isoquinolyl, thienyl, imidazolyl, thiazolyl, indolyl (e.g., 1H-indolyl), pyrroyl, oxazolyl, benzofuryl, benzothienyl, benzthiazolyl, isoxazolyl, pyrazolyl, triazolyl, tetrazolyl, indazolyl (e.g., 1H-indazolyl), 1,2,4-thiadiazolyl, isothiazolyl, purinyl, carbazolyl, benzimidazolyl, indolinyl, benzodioxolanyl, and benzodioxane. Heteroaryl groups may be substituted or unsubstituted. The nitrogen atom may be substituted or unsubstituted (i.e., N or NR wherein R is H or another substituent, if defined). The nitrogen and sulfur heteroatoms may optionally be oxidized (i.e., N→0 and S(O)ₚ, wherein p is 0, 1 or 2).

The term “halo” or “halogen” includes fluoro, chloro, bromo and iodo.

As used herein, the term “substituted” refers to at least one hydrogen atom that is replaced with a non-hydrogen group, provided that normal valencies are maintained and that the substitution results in a stable compound. When a group is noted as “optionally substituted”, the group may or may not contain non-hydrogen substituents. When present, the substituent(s) may be selected from alkyl, halo (e.g., chloro, bromo, iodo, fluoro), hydroxyl, alkoxy, oxo, alkanoyl, aryloxy, alkanoyloxy, amino (—NH₂), alkylamino (—NHalkyl), cycloalkylamino (—NHcycloalkyl), arylamino (—NHaryl), arylalkylamino (—NHarylalkyl), disubstituted amino (e.g., in which the two amino substituents are selected from alkyl, aryl or arylalkyl, including substituted variants thereof, with specific mention being made to dimethylamino), alkanoylamino, aroylamino, arylalkanoylamino, thiol, alkylthio, arylthio, arylalkylthio, alkylthiono, arylthiono, arylalkylthiono, alkylsulfonyl, arylsulfonyl, arylalkylsulfonyl, sulfonamide (e.g., —SO₂NH₂), substituted sulfonamide (e.g., —SO₂NHalkyl, —SO₂NHaryl, —SO₂NHarylalkyl, or cases where there are two substituents on one nitrogen selected from alkyl, aryl, or alkylalkyl), nitro, cyano, carboxy, unsubstituted amide (i.e. —CONH₂), substituted amide (e.g., —CONHalkyl, —CONHaryl, —CONHarylalkyl or cases where there are two substituents on one nitrogen selected from alkyl, aryl, or alkylalkyl), alkoxycarbonyl, aryl, guanidine, heterocyclyl (e.g., pyridyl, furyl, morpholinyl, pyrrolidinyl, piperazinyl, indolyl, imidazolyl, thienyl, thiazolyl, pyrrolidyl, pyrimidyl, piperidinyl, homopiperazinyl), and mixtures thereof. The substituents may themselves be optionally substituted, and may be either unprotected, or protected as necessary, as known to those skilled in the art, for example, as taught in Greene, et al., “Protective Groups in Organic Synthesis”, John Wiley and Sons, Second Edition, 1991, hereby incorporated by reference in its entirety.

Unless otherwise noted, the present disclosure is intended to include all isotopes of the samples used herein.

Aspects of the present disclosure are directed to a surface-enhanced Raman scattering (SERS) substrate and system, e.g., a preferably liquid matrix containing suspended particles. The system may be used without a supportive substrate.

The SERS substrate and system can detect a benzamide compound with an aqueous solution detection of as low as from 1×10⁻¹⁴ to 1×10⁻¹⁰ molar (M). In some embodiments, the benzamide compound has a formula (I)

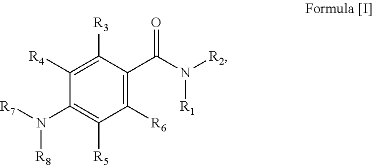

wherein R₁, R₂, R₇, and R₈ are each independently selected from the group consisting of a hydrogen atom, an optionally substituted alkyl, an optionally substituted aryl, and an optionally substituted heterocyclic aryl; and wherein R₃, R₄, R₅, and R₆ are each independently selected from the group consisting of a hydrogen atom, a halogen atom, an optionally substituted alkyl. In some embodiments, at least one of R₁ and R₂; and R₇ and R₈ is hydrogen. In a preferred embodiment, R₃, R₄, R₅, and R₆ are each independently hydrogen. In a preferred embodiment, the benzamide compound is a procainamide, e.g., N-acetylprocainamide or NAPA.

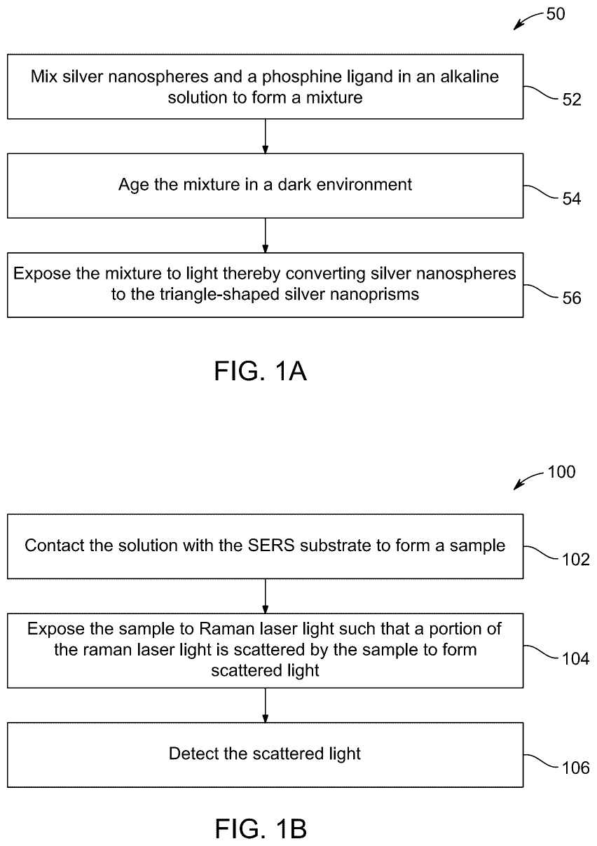

Referring to FIG. 1A, a schematic flow chart of a method 50 of preparing the triangle-shaped silver nanoprisms is described. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes mixing silver nanospheres and a ligand, preferably a phosphineligand, in an alkaline solution to form a mixture. The phosphine ligand is at least one of a bis (p-sulfonatophenyl) phenylphosphine dihydrate dipotassium (BSPP) salt, and a triphenylphosphine-3,3′,3″-trisulfonic acid trisodium salt. In a preferred embodiment, the phosphine ligand is BSSP. In some embodiments, the silver nanospheres have an average particle size in a range of 20 to 120 nm, or preferably 30 to 110 nm, preferably 40 to 100 nm, preferably 50 to 90 nm, or preferably 60 to 80 nm, or preferably 65 to 75 nm. The silver nanospheres and the phosphine ligand are mixed in an alkaline solution. The molar ratio of the silver nanospheres to the phosphine ligand in the alkaline solution is in a range of 2:1 to 1:2, preferably 1:1. One of the factors affecting the shape of the silver nanoprisms is the pH. For this reason, the silver nanospheres to the phosphine ligand are mixed in the alkaline solution, including at least one pH-adjusting agent selected from LiOH, NaOH, KOH, and Ca(OH)₂, preferably NaOH. The pH of the mixture is adjusted to 9.5-11, preferably between 10-11.

At step 54, the method 50 includes optionally aging the mixture in a dark environment. In an embodiment, the mixture is aged for 10-20 hours, preferably 12-18 hours, preferably 14-16 hours, preferably 15 hours.

At step 56, method 50 includes exposing the mixture to light, thereby converting silver nanospheres to triangle-shaped silver nanoprisms. The light may be any suitable source for emitting radiation at the desired wavelength. For example, commercially available semiconductor lasers, helium-neon lasers, carbon dioxide lasers, light emitting diodes (LED), incandescent lamps, and many other known radiation emitting sources may be used as the excitation radiation source. In a specific embodiment, the light source is a monochromatic LED light having a wavelength of from 400 to 500 nm under a powder of from 120 to 180 watts (W), or preferably a wavelength of from 420 to 480 nm under a powder of from 130 to 170 W, or preferably a wavelength of from 440 to 460 nm under a powder of from 140 to 160 W, or preferably a wavelength of from 445 to 455 nm under a powder of from 145 to 155 W. In a specific embodiment, the light source is the monochromatic LED light having a wavelength of about 455 nm under a powder of about 150 W. In some embodiments, wavelengths beyond this range may be selected as well, the selection being dependent on the desired morphology, and this selection may be obvious to a person skilled in the art.

The triangle-shaped silver nanoprisms of the present disclosure have an average particle size of 70 to 120 nanometers (nm), preferably about 80 to 110 nanometers (nm), preferably about 90 to 100 nanometers (nm), or preferably about 95 nm. The triangle-shaped silver nanoprisms are preferably monodisperse.

FIG. 1B illustrates a schematic flow chart of a method 100 of obtaining a Raman spectrum of an analyte in a solution. In a preferred embodiment, the analyte is N-acetyl procainamide (NAPA), and the solution is human blood. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes contacting the solution with the SERS substrate to form a sample. The solution includes benzamides or the benzamide compound of formula (I). Suitable examples of benzamide compounds include, but are not limited to, ethenzamide, salicylamide, salverine, procainamide, moclobemide, alizapride, batanopride, bromopride, cinitapride, cisapride, clebopride, dazopride, itopride, metoclopramide, mosapride, prucalopride, renzapride, trimethobenzamide, veralipride, zacopride, azapride, amisulpride, levosulpiride, nemonapride, remoxipride, sulpiride, sultopride, tiapride, bromadoline, 3-aminobenzamide, N-acetylprocainamide, aminohippuric acid, chidamide, denipride, entinostat, eticlopride, imatinib, mocetinostat, procarbazine, raclopride, sunifiram, and/or combinations thereof. In a specific embodiment, the benzamide compound is N-acetylprocainamide. The analyte is dispersed in the solution. The solution may be blood, milk, urine, or plasma, preferably human blood and preferably an aqueous solution that may contain organic or cellular contaminants.

At step 104, the method 100 includes exposing the sample to Raman laser light such that a portion of the Raman laser light is scattered by the sample to form scattered light. In some embodiments, the scattered light is monitored from 400-2,000 cm⁻¹, or preferably 450-1,950 cm⁻¹, or preferably 500-1,900 cm⁻¹, or preferably 550-1,850 cm⁻¹, or preferably 600-1,800 cm⁻¹, or preferably 650-1,750 cm⁻¹, or preferably 500-1,900 cm⁻¹, or preferably 700-1,700 cm⁻¹, or preferably 800-1,650 cm⁻¹, or preferably 900-1,600 cm⁻¹, or preferably 1,000-1,550 cm⁻¹, or preferably 1,100-1,500 cm⁻¹, or preferably 1,200-1,450 cm⁻¹, or preferably 1,300-1,400 cm⁻¹ for detecting a benzamide compound having a detection limit in the range of 10⁻⁴ to 10⁻¹⁴ M, preferably 10⁻⁵ M, preferably 10⁻⁶ M, preferably 10⁻⁷ M, preferably 10⁻⁸ M, preferably 10⁻⁹ M, preferably 10⁻¹⁰ M, preferably 10⁻¹¹ M, preferably 10⁻¹² M, preferably 10⁻¹³ M.

At step 106, the method 100 includes detecting the scattered light. The scattered light may be detected using various software or methods known in the art, such as nephelometry or turbidimetry. In some embodiments, the method further includes quantifying the amount of the analyte present in the solution based on the intensity of the scattered light. The intensity of the scattered light is indicative of the concentration of the analyte in the solution. In other words, a higher intensity of scattered light is indicative of a greater concentration of the analyte in the solution. Although, the examples provided herein refer to the use of the SERS substrate for detection of NAPA, the SERS substrate of the present disclosure may be used for detection of other benzamides as well.

In a specific embodiment, when the analyte is NAPA, the intensity of the scattered light linearly correlates with the amount of NAPA present in the solution. The linear dynamic range of NAPA present in the solution is from 0.5×10⁻¹² to 0.5×10⁻⁴ M, or preferably 0.5×10⁻¹¹ to 0.5×10⁻⁵ M, or preferably 0.5×10⁻¹⁰ to 0.5×10⁻⁶ M, or preferably 0.5×10⁻⁹ to 0.5×10⁻⁷ M, or preferably 0.5×10⁻⁷ to 0.5×10⁻⁹ M, or preferably 0.5×10⁻⁶ to 0.5×10⁻¹⁰ M, or preferably 0.5×10⁻⁵ to 0.5×10⁻¹¹ M. The SERS substrate of the present disclosure demonstrates sensitivity for NAPA in aqueous solutions with a detection limit of 0.5×10⁻¹³ M, corresponding to excellent recovery and stability.

### EXAMPLES

The following examples demonstrate a surface-enhanced Raman scattering (SERS) substrate (e.g., system of suspended particles) for detecting analytes such as N-acetyl procainamide (NAPA), a method of forming the SERS substrate, and a method of obtaining a Raman spectrum of an analyte using the SERS substrate, as described herein. The examples are provided solely for illustration and are not to be construed as limitations of the present disclosure, as many variations thereof are possible without departing from the spirit and scope of the present disclosure.

### Example 1: Materials

All materials and chemicals utilized in this work were used without further modifications. Trisodium citrate (Na₃C₆H₅O₇), silver nitrate (NaNO₃), N-Acetyl procainamide (NAPA), and citric acid (C₆H₈O₇) were purchased from Sigma Aldrich. Bis(p-sulfonatophenyl) phenyl-phosphine dihydrate (BSPP), sodium tertrahydroborate (NaBH₄), and sodium hydroxide (NaOH) were purchased from Alfa Aesar, USA. Throughout all of the tests, Milli-Q grade (mQ) water (>18 M) was utilized for solution preparation.

### Example 2: Synthesis of Silver Nanospheres and Silver Nanoprisms

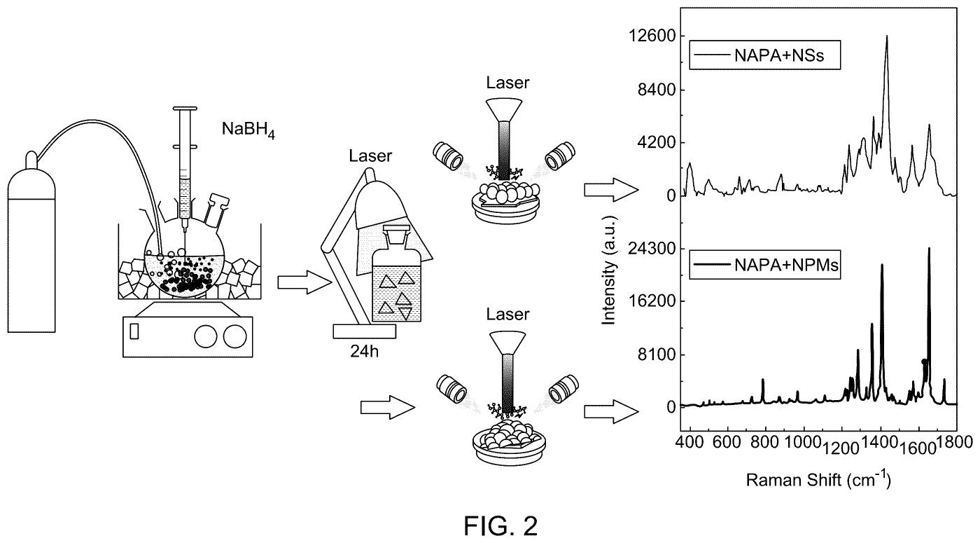

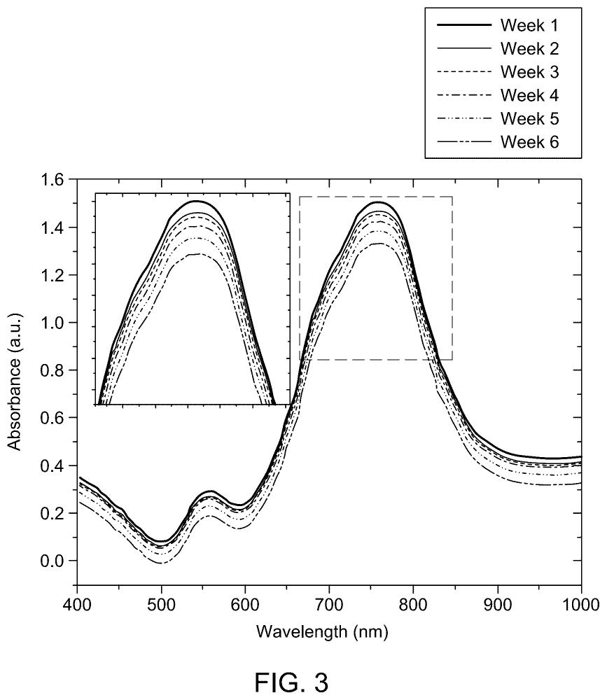

Colloidal silver nanosphere was prepared [See: N. Mahar, A. Al-Ahmed, A. A. Al-Saadi, Synthesis of vanadium carbide MXene with improved inter-layer spacing for SERS-based quantification of anti-cancer drugs, Appl Surf Sci. 607 (2023), 155034; and N. Mahar, A. A. Al-Saadi, SERS-Based Ultralow Concentration Detection of Anticancer Gemcitabine Using Size-Controlled Silver Nanoparticles, Arabian J. Sci. Eng. 47 (2022) 7197-7205, incorporated herein by reference in its entirety] and modified to prepare the nanoprisms following a modified method [See: L. Mikac, M. Ivanda, M. Gotić, T. Mihelj, L. Horvat, Synthesis and characterization of silver colloidal nanoparticles with different coatings for SERS application, J. Nanoparticle Res. 16 (12) (2014) 1-13, incorporated herein by reference in its entirety]. Applying a typical synthesis procedure (FIG. 2), in a 250 mL 3-neck flask, 95 mL of mQ water was added, followed by N₂ purging for 40 min on an ice bath with constant stirring at 500 RPM. Then 2 mL of (0.10 M) AgNO₃ was injected along with 1 mL (0.17 M) sodium citrate, followed by 1 mL of cooled (50 mM) NaBH₄ was injected resulting in a chemical reduction and pale-yellow appearance of the mixture. After 20 min of stirring an ice bath in the mixture, 0.5 mL of additional NaBH₄ was injected (5 min intervals), followed by the addition of 1 mL of BSPP to efficiently reduce and cap with negative charges on the silver nanosphere to avoid early agglomeration. To adjust the pH (9.5-11), the 0.2 M NaOH was added dropwise as the higher pH(>10) and a [BSPP]/[Ag⁺] ratio close to 1 are used for the synthesis of a monodisperse triangular bipyramidal silver [See: Knaapila et al. Structural Study of the Photo-Mediated Growth of Silver Nanoprisms, Molecules 25 (2020), incorporated herein by reference in its entirety]. Then the mixture is kept in the dark in a closed container for 15 h for the aging of the solution. After, the solution was irradiated for a photoreaction to convert the nanospheres into nanoprisms in the dark with monochromatic LED light of 455 nm wavelength and 150 W of power for 24 h. The selection of a specific LED wavelength depends on the desired size of the seed as well as the targeted morphology. By increasing the wavelength, the dipolar absorption decreases with an increase in the longitudinal plasmon modes, and the 455-nm wavelength was noticed to favor the truncated geometry. For the nanoprisms morphology, the favorable size of the seed is between 20 and 120 nm, therefore, to attain the targeted morphology, a wavelength-specific LED light source was employed [See: Scardaci et al. Monochromatic light driven synthesis and growth of flat silver nanoparticles and their plasmon sensitivity, J Mater Chem C Mater. 8 (2020) 9734-9741; Stamplecoskie et al. Light emitting diode irradiation can control the morphology and optical properties of silver nanoparticles, J Am Chem Soc. 132 (2010) 1825-1827, incorporated herein by reference in its entirety]. The resulting product was green-colored nanoprisms with an average size of 95.2 nm. The prepared AgNPMs solutions (FIG. 3) were used for the SERS study without further treatment. They showed stability for a one-month duration, as depicted by the measured excitation spectrum in which the peak consistency proves stability.

### Example 3: Raman and SERS Measurements

The shape transformation and morphology of prepared AgNSs and AgNPMs were compared using a variety of characterization techniques, such as a UV-visible spectrophotometer. Both Raman and SERS spectra were collected using a LabRAM HR evolution spectrometer with a He—Ne 17 mW power laser source at 633 nm wavelength excitation. An objective lens of 50× power and 1800 mm switching grating at an acquisition time of 20 seconds was selected for both the Raman and SERS spectra. The SERS study to quantify NAPA was conducted in an aqueous solution at a concentration range from 10⁻³ to 10⁻¹⁴ M by a subsequent dilution method in mQ water. The prepared solutions were used without further treatment. The v/v ratio of the NAPA probe to the substrate (AgNSs and AgNPMs) was set (2:1), and all the spectra were collected at room temperature. The results are an average of 3 runs of each spectrum. The enhancement factor (EF) was determined using the formula:

$$E F = ( {I}_{SERS} / {I}_{Raman} ) \times ( {C}_{Raman} / {N}_{SERS} )$$

where I_{SERS} and I_{Raman} are the intensities of probe molecules in the laser-illuminated area in both SERS (23585 a.u.) and normal Raman (1505 a. u.) experiments, and C_{Raman} (10⁻⁴ M) and N_{SERS} (10⁻¹² M) are the concentrations of the characteristic Raman and SERS peaks of the analyte, respectively.

The UV-Vis was taken on Agilent, with standard quartz cuvettes used to collect the electronic spectra. A JEOL-USA JEM-2100F field emission microscope was used to capture the morphological characteristics at 200 kV accelerating voltage.

### Results and Discussion

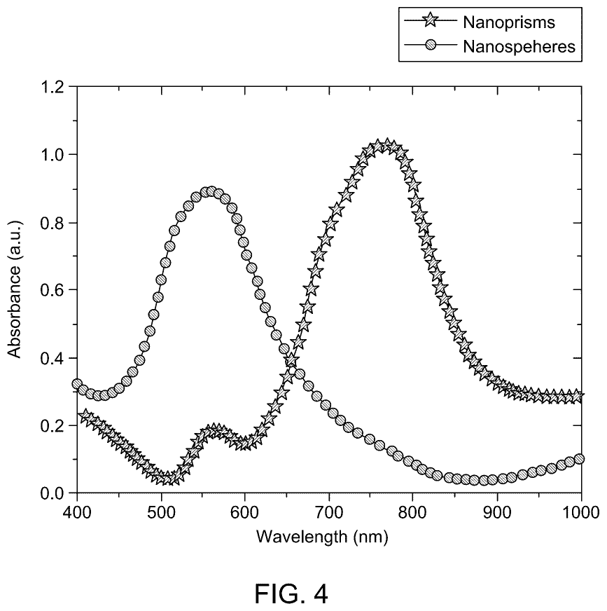

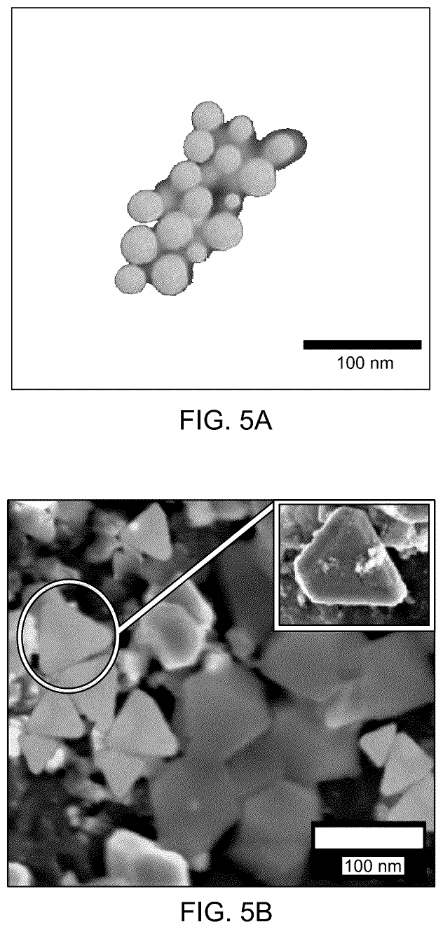

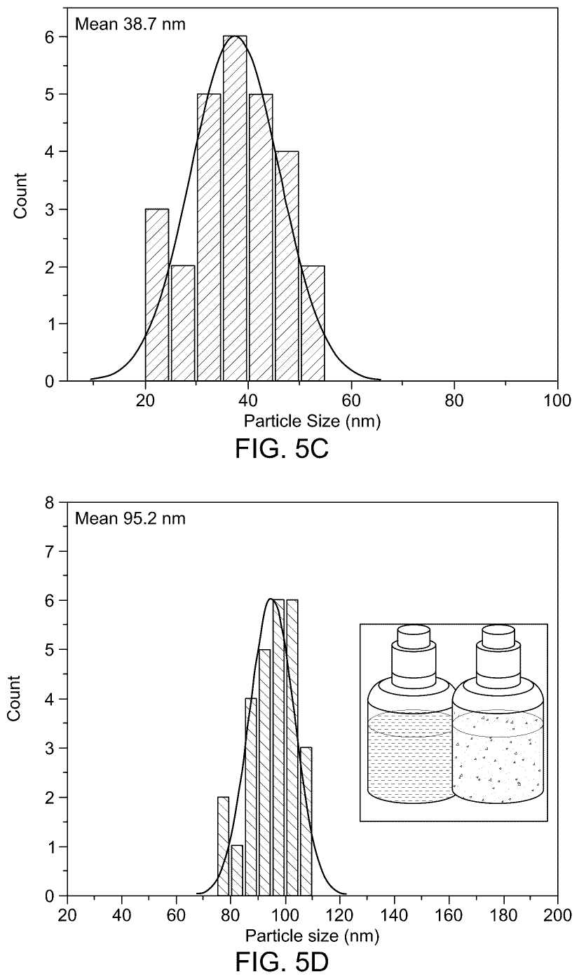

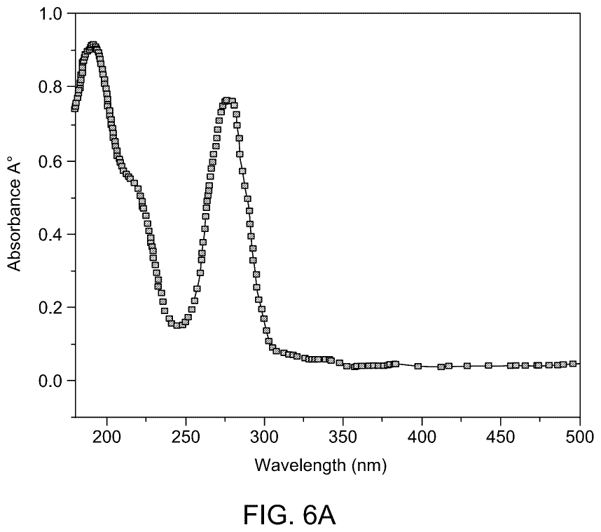

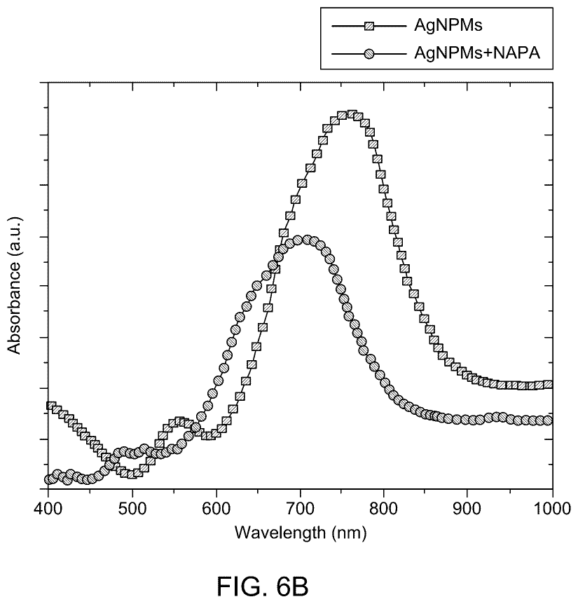

The UV/vis-NIR spectra of prepared nanoparticles are shown in FIG. 4. It can be seen (FIG. 4) that a characteristic absorption band at 569 nm was observed with an average size of 38.7 nm (FIG. 5C). The observed red shift in the band position as compared to the reported literature value of 400 nm could be attributed to size analogy and agglomeration. After the photo-induced shape transformation of AgNSs seed into a nanoprisms morphology, a concomitant rise of a new band at 769 nm is the in-plane dipole peak associated with the large bimodal distributed prisms with 95.2 nm edged-length (FIG. 5). There is a small band observed at 569 nm attributed to the out of plane dipole peak of the nanoprisms, a characteristic feature of the bimodal growth. Since the AgNPMs are coated by negatively charged BSPP and citrates ligands, their components should repel each other, but the irradiated light induces the optical attractive forces which resulted in a pronounced dipole plasmon resonance [See: Blaber, A. I. Henry, J. M. Bingham, G. C. Schatz, R. P. Van Duyne, LSPR imaging of silver triangular nanoprisms: Correlating scattering with structure using electrodynamics for plasmon lifetime analysis, J. Phys. Chem. C 116 (2012) 393-403, incorporated herein by reference in its entirety]. As a result, a redshift in the exciton wavelength was observed in the case of AgNPMs. Besides this photoexcitation process, the edges and the tips of the nanoprisms are extremely polarized resulting in overcoming the electrostatic repulsion and leading to a fusion of a few types of nanoprisms, hence the average size of the nanoprisms was enhanced, (FIG. 5B) [See. Tang, et al. Polarized SERS substrates with directionality, repeatability and orderability: an anisotropic Ag nanocavity array, J Mater Chem C Mater. 10 (2022) 14549-14559, incorporated herein by reference in its entirety]. The interaction of the probe molecule (NAPA) could be observed in the excitation spectrum depicted in FIG. 6B, where a blue shift in a band position from 769 to 713 nm accompanied by a change in its intensity and rise of small shoulders was observed, indicating that a photo-induced charge transition took place between the substrate and the NAPA molecules at hot spots. FIG. 6A shows the UV-Vis spectra of free NAPA molecules.

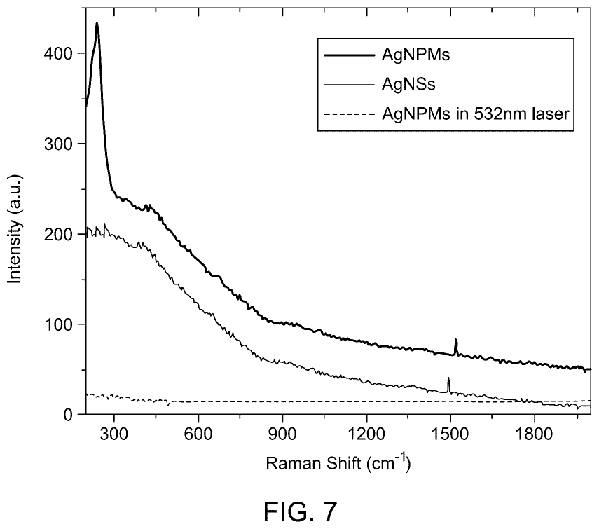

Surface characterization was carried out using a scanning electron microscope (SEM) following conventional methods as known in the literature. FE-SEM images, shown in FIG. 5B, confirms that the nanospheres have been successfully converted into nanoprisms with various sizes and shapes, possibly due to a photo-induced fusion. The majority of the thermally synthesized nano-particles have a diameter ranging between 35 and 40 nm and are spherical in nature (FIG. 5A). After being exposed to light for 24 h, AgNSs with a mean particle size of 38.7 nm transformed into a nanoprism-based structure due to photoinduced plasmonic truncation associated with a change in color from yellow to green with an average size of 95 nm as depicted in FIG. 5B. Categorically, photoexcitation plays multiple roles in seed growth from sphere to nanoprisms, as it initiates the formation of enlarged length-edged nanoparticles and excites the dipole plasmon band of the formed nanoprisms. That resulted in the fusion of the AgNPMs and, hence, deformed the uniform sphere-shaped nanoparticles. The Raman spectra of the prepared nanoparticles (FIG. 7) show no Raman peaks at 532 nm laser excitation though this wavelength is close to the SPR band (569 nm) of the silver nanospheres. However, there are a few less intense peaks and shoulders, along with an intense band at 288 cm⁻¹. A shift in the band position was observed in the case of the 633 nm laser source. This could be attributed to the photoinduced aggregation to generate the local hot spots.

The uniform and unique patterns of the Raman spectra provide a reliable means to identify the fingerprints of biomolecules and active drug contents. SERS-based techniques are particularly useful to detect very low concentrations of bioactive components. There are two acceptable concepts promoting Raman signal enhancements: the electromagnetic (EM) and chemical (CM) mechanisms. The EM can lead to an enhancement factor of up to 10⁸ owing to the longitudinal localized surface plasmonic resonance (LLSPR) emanated when the probe molecules are trapped within hot spots in the range of a few nanometers (4-10 nm) distances. The analyte molecules position themselves within hotspots filling small gaps in the vicinity with noble metal nanoparticles. Several analytical protocols, a summary of which is shown in Table 1, were developed, and reported for the detection of the drug NAPA.

TABLE 1
Limits of detection (LODs) and linear dynamic ranges
(LDRs) of some established techniques for the quantification
of NAPA compared to this work.

| Method | LDR | LOD | | R2 |
|---|---|---|---|---|
| TLC* | 1-5 μg/ml | 60 | ng/ml | — |
| GL** | — | 10 | ng/ml | — |
| HPLC*** | — | 2-10 | mg | — |
| RP-LC**** | — | 10 | ng | — |

| | | | |
|---|---|---|---|
| UV-HPLC***** | 0.1-8 | 0.1 | 0.99 |
| SERS | 0.5 × 10⁻⁴ to 0.5 × 10⁻¹²M | 0.5 × 10⁻¹³M | 0.945 |

[See: *Hadzija et al. Quantitative thin-layer chromatographic method for the determination of procainamide and its major metabolite in plasma, J Chromatogr B Biomed Sci Appl. 143 (1977) 307-313;
**Kark et al. Thin-layer chromatographic determination of procainamide and N-acetylprocainamide in human serum and urine at single-dose levels, J Chromatogr B Biomed Sci Appl. 277 (1983) 261-272;
***Stearns, et al. Determination of procainamide and N-acetylprocainamide by “high-performance liquid chromatography Ix1 Determination of Procainamide and N-Acetylprocainamide by ‘High-Performance’ Liquid Chromatography, Clin. Chem. 27 (27) (1981) 2064-2067;
****Coyle et al. Reversed-Phase Liquid Chromatography Method for Measurement of Procainamide and Three Metabolites in Serum and Urine: Percent of Dose Excreted as Deethyl Metabolites, J Pharm Sci. 76 (1987) 402-405;
*****Carr et al. Simultaneous quantification of procainamide and n-acetylprocainamide with high-performance liquid chromatography, J Chromatogr A. 129 (1976) 363-368].

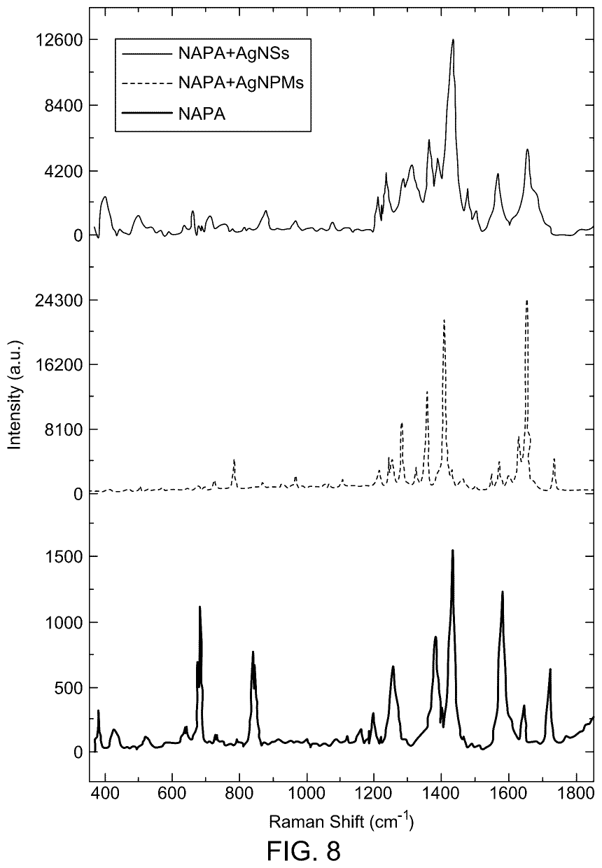

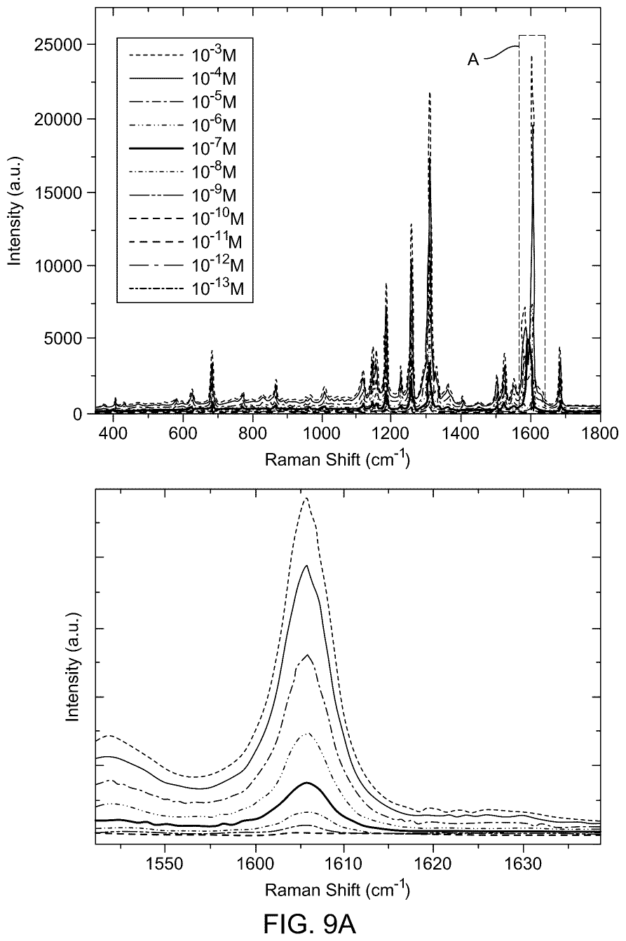

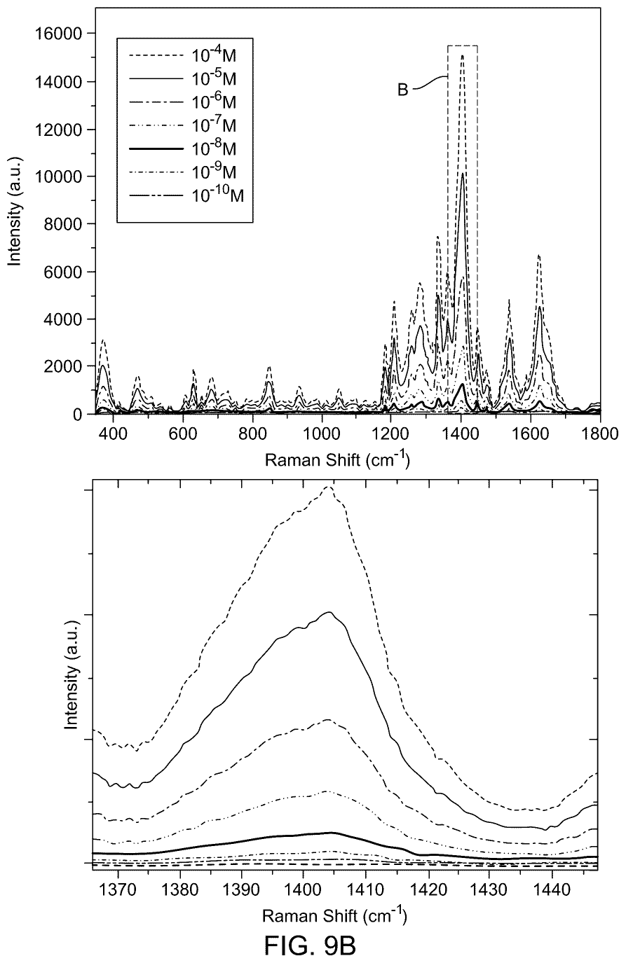

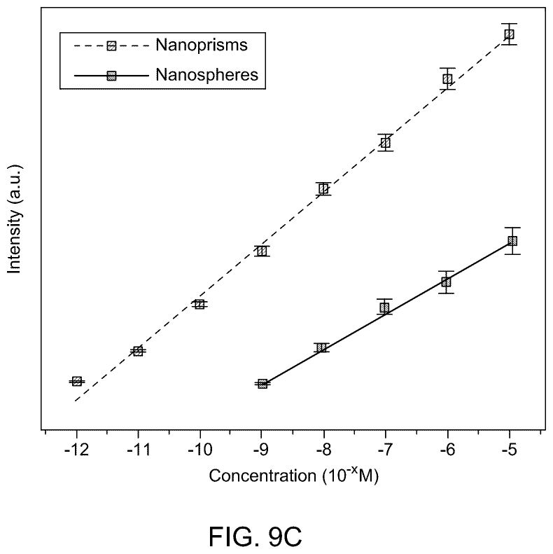

Achieving an even lower detection limit with a reproducible method is still an area of interest. A comparison of SERS and Raman spectra of NAPA with both the AgNSs and NPMs substrates is displayed in FIG. 8. Eight distinct Raman signals of NAPA between 600 and 1800 cm⁻¹ spectral range of wavelength shifts were identified in FIG. 3. Notably, the five prominent Raman bands at 680 cm⁻¹ (assigned to C-N-C bending), 1431 cm⁻¹ (C—C stretching), 1576 cm⁻¹ (NH₂ bending), 1643 cm⁻¹ (C—C stretching), and 1719 cm⁻¹ (C═O stretching) encountered notable changes in terms of spectral intensities as well as wavenumber shifts after the SERS phenomenon (FIG. 8). Such changes occurred due to shape and high symmetry, which results in more hot spots, hence strong EM and chemical interactions, gesturing the role of the photo-induced charge transfer, LLSPR, and hotspot characteristics. For instance, shifts in peak positions were noted in the case of AgNPMs substrate for the Raman lines centered at 1719 cm⁻¹ (shifted to 1733 cm⁻¹) and 1643 (shifted to 1652 cm⁻¹), while redshifts were encountered with the decrease in peak intensity at 1576 cm⁻¹ (shifted to 1568 cm⁻¹), and 1431 cm⁻¹ (shifted to 1407 cm⁻¹). Likewise, for the case of AgNSs, NAPA peaks appearing in 1719, 1576, 1431, and 680 cm⁻¹ were shifted to 1721, 1578, 1432, and 682 cm⁻¹, respectively, while the C═O stretching band at 1643 cm⁻¹ demonstrated a red-shift to 1654 cm⁻¹. By analyzing the SERS spectra at various drug concentrations in the range of 10⁻⁴-10⁻¹⁴ M, a limit of detection (LOD) of NAPA was successfully achieved to be in orders of 10⁻¹³ M in the case of AgNPMs and 10⁻⁹ M for AgNSs (Table 2 and FIG. 9A).

TABLE 2
Comparison of the limit of detection (LOD), linear dynamic
range (LDR), and Raman intensity enhancement factor
(EF) parameters using AgNSs and AgNPMs materials as
active substrates for sensing of NAPA drug.

| SERS Substrate | LOD | LDR | R² | EF |
|---|---|---|---|---|
| AgNSs | 0.5 × 10⁻⁹M | 10⁻⁵-10⁻⁹M | 0.9172 | 1.21 × 10⁵ |
| AgNPMs | 0.5 × 10⁻¹³M | 10⁻⁴-10⁻¹²M | 0.9456 | 1.57 × 10⁸ |

The decline in peak intensity as a result of the change in NAPA concentrations follows a consistent SERS spectral pattern. The decline in peak intensity as a result of the change in NAPA concentrations follows a consistent SERS spectral pattern. Since the signal strength is directly proportional to the analyte concentration, it falls linearly as concentration [See: Wang et al. Shell thickness-dependent Au@Ag nanoparticles aggregates for high-performance SERS applications, Talanta 195 (2019) 506-515; Ghopry et al. Nanoparticle/WS2 Nanodome/Graphene van der Waals Heterostructure Substrates for Surface-Enhanced Raman Spectroscopy, ACS Appl Nano Mater. 3 (2020) 2354-2363]. The results show that the intensities can be correlated with the drug analyte's logarithm of concentration with correlation coefficients of 0.9456 and 0.9173 for AgNPMs (Raman peak at 1652 cm⁻¹) and AgNSs (Raman peak at 1432 cm⁻¹), respectively. Furthermore, the peak at 1652 cm⁻¹ had a wide linear dynamic range (LDR) of 10⁻⁴ to 10⁻¹³ M for AgNPMs (FIG. 9B), whereas the AgNSs (1432 cm⁻¹) had a relatively narrower one in comparison to the first band (FIG. 9C). While the wider spectral bandwidths reflect a stronger interaction in the case of NSs with NAPA (FIG. 8), the Raman signal intensity enhancement when the NPMs substrate is employed was observed multiple folds stronger, which could be explained in terms of the anisotropic morphology of NPMs due to the photo-induced truncation and lightning rod effect. Having three double-edged sharp vertices/tips of ˜60 nm is believed to significantly contribute to the nanoprisms superior optical and electronic properties. That consequently led to a strong plasmonic influence to spectroscopically detect the low concentrations of probe molecules.

To quantify the enhancement efficiency, Raman enhancement factors (EF) were computed using the same aforementioned reference peaks. While the EF in Raman signals reached up to 1.21×10⁵ in the case of AgNSs-NAPA interaction, the AgNPMs-NAPA system exhibited a further better EF of the value of 1.57×10⁸ (Table 2). The variation in the nanostructures between the two nanoparticles is believed to be the main reason behind the difference in their SERS performance, although both contributed notably to the intensity enhancement process. A possible explanation for AgNPMs exhibiting significantly more enhancement than AgNSs is due to the light-induced fusion of AgNPMs, which results in an aggregation of four orders of magnitude and a decreased distance between the analyte molecules and hotspots, which is not the case with AgNSs. Selectivity and Stability Studies

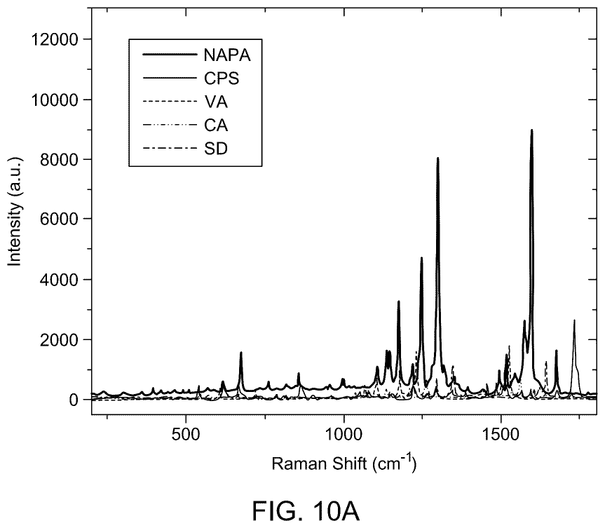

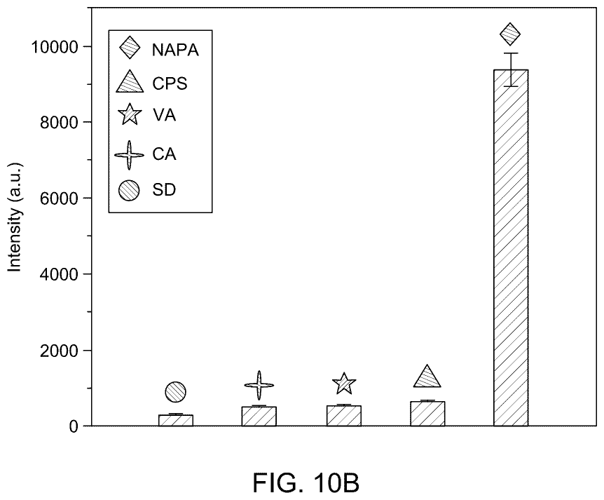

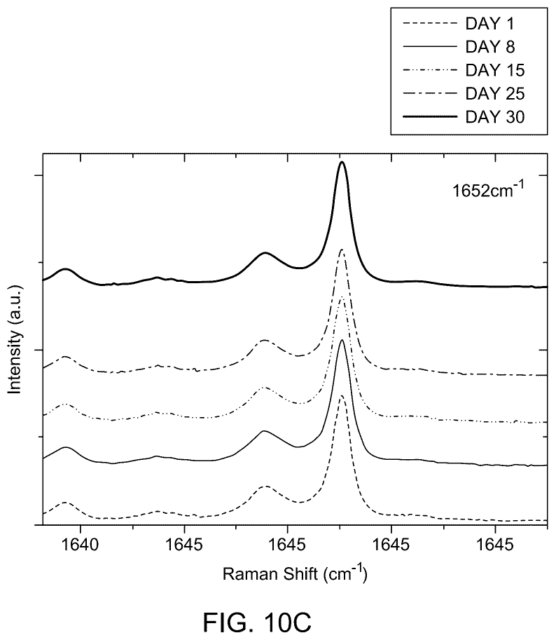

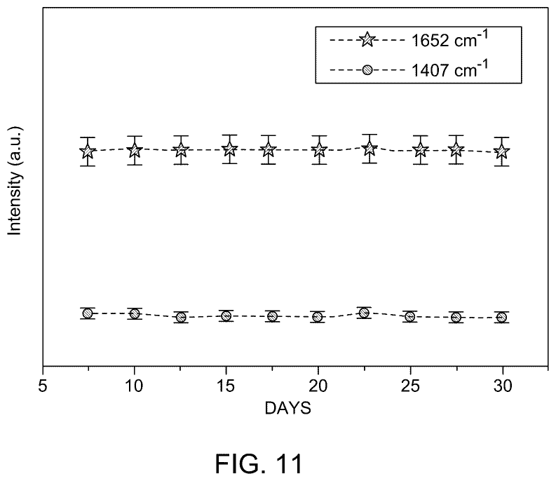

The stability of the synthesized materials and the reproducibility of their SERS performance were also tested. The repeatability of the method is demonstrated, for instance, by the Raman intensity distribution of the peak 1652 cm⁻¹ during the studied period of time, which demonstrated an adequate consistency with an RSD of 1.89%. Likewise, the selectivity of the approach was tested by considering four distinct interferents, namely sulindac (SD), ciprofloxacin (CPS), citric acid (CA), and valeric acid (VA), 10⁻² M, in 1000 times higher concentration than the analyte and mixing them with a NAPA solution at a concentration of 1×10⁻⁶ M. The other bioactive species' interference can be seen to have little effect on the SERS spectra of the NAPA solution (FIGS. 10A-10B). The average recovery was estimated to be 97%, while the RSD of the SERS peaks for the concentration of 1×10⁻⁶ M was found to be 1.45%. In a test for stability, the findings shown in FIG. 10C indicated that Raman peak intensity at 1652 and 1407 cm⁻¹ barely changed throughout the course of a four-week period (702-710) for the period of 30 days (FIG. 11). It is obvious that the peak intensity is persistent with negligible shift and change in peak pattern.

To summarize, SERS substrates containing AgNPMs were prepared and used for analyte detection using Raman spectra. The synthesized nanoprisms were characterized by FE-SEM, UV-Vis, and Raman spectroscopy, which confirmed transformation in the morphology of the prepared nanomaterials from nanospheres. The uniform and unique patterns of the Raman spectra provided a reliable means to identify the fingerprints of biomolecules and active drug contents. SERS-based techniques are particularly useful for detecting very low concentrations of bioactive components, which was evident from various concentrations of NAPA solutions ranging from 10⁻⁴ to 10⁻¹⁴ M were examined, and a detection limit of 0.5×10⁻¹³ M was successfully accomplished using the reference Raman peak at 1652 cm⁻¹, associated with the C—C stretching modes (Table 1, Table 2). The examples demonstrated better sensitivity and SERS efficiency of the photo-induced AgNPMs as compared to AgNSs analogs. The prepared nanoprisms have the potential for long-term usage in the field of drug sensing, pharmacology, and other laboratory-based diagnoses.

Numerous modifications and variations of the present disclosure are possible in light of the above teachings. It is therefore to be understood that within the scope of the appended claims, the invention may be practiced otherwise than as specifically described herein.

1. A surface-enhanced Raman scattering (SERS) system, comprising:
monodisperse triangle-shaped silver nanoprisms (AgNPMs),
wherein the AgNPMs are suspended in a liquid matrix, or, optionally, at least partially coat a transparent substrate;
wherein the triangle-shaped silver nanoprisms are prepared by:
mixing silver nanospheres and a phosphine ligand in an alkaline aqueous solution to form a mixture;
aging the mixture in a dark environment; and
exposing the mixture to laser light at a wavelength of 455 nm ±5 nm thereby converting the silver nanospheres to the triangle-shaped silver nanoprisms.

2. The SERS system of claim 1, wherein the triangle-shaped silver nanoprisms are supported on a glass substrate selected from the group consisting of a fluorine doped tin oxide (FTO) coated glass substrate, a tin doped indium oxide (ITO) coated glass substrate, an aluminum doped zinc oxide (AZO) coated glass substrate, a niobium doped titanium dioxide (NTO) coated glass substrate, an indium doped cadmium oxide (ICO) coated glass substrate, an indium doped zinc oxide (IZO) coated glass substrate, a fluorine doped zinc oxide (FZO) coated glass substrate, a gallium doped zinc oxide (GZO) coated glass substrate, an antimony doped tin oxide (ATO) coated glass substrate, a phosphorus doped tin oxide (PTO) coated glass substrate, a zinc antimonate coated glass substrate, a zinc oxide coated glass substrate, a ruthenium oxide coated glass substrate, a rhenium oxide coated glass substrate, a silver oxide coated glass substrate, and a nickel oxide coated glass substrate.

3. The SERS system of claim 1, wherein the triangle-shaped silver nanoprisms have an average particle size of from 70 to 120 nanometers (nm), and
wherein the SERS substrate can detect a benzamide compound with a detection of from 1×10⁻¹⁴ to 1×10⁻¹⁰ molar (M).

4. The SERS system of claim 3, wherein the triangle-shaped silver nanoprisms have an average particle size of about 95 nm.

5. The SERS system of claim 1, wherein the liquid matrix comprises a benzamide compound of formula (I) 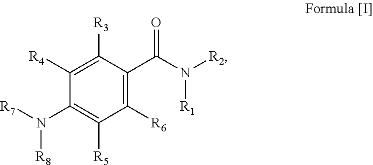
wherein R₁, R₂, R₇, and R₈ are each independently selected from the group consisting of a hydrogen atom, an optionally substituted alkyl, an optionally substituted aryl, and an optionally substituted heterocyclic aryl; and
wherein R₃, R₄, R₅, and R₆ are each independently selected from the group consisting of a hydrogen atom, a halogen atom, an optionally substituted alkyl.

6. The SERS system of claim 5, wherein the benzamide compound is N-acetylprocainamide (NAPA).

7. The SERS system of claim 1, wherein the benzamide compound is NAPA, and wherein the SERS substrate has a detection limit of 0.5×10⁻¹³ M.

8. A method of forming a SERS substrate, the method comprising:
preparing triangle-shaped silver nanoprisms (AgNPMs) by:
mixing silver nanospheres and a phosphine ligand in an alkaline aqueous solution to form a mixture;
aging the mixture in a dark environment; and
exposing the mixture to laser light having a wavelength of 455 nm±5 nm and thereby converting the silver nanospheres to the triangle-shaped silver nanoprisms.

9. The method of claim 8, wherein the silver nanospheres have an average particle size in a range of 20 to 120 nm.

10. The method of claim 8, wherein the phosphine ligand is at least one of a bis(p-sulfonatophenyl) phenylphosphine dihydrate dipotassium (BSPP) salt, and a triphenylphosphine-3,3′,3″-trisulfonic acid trisodium salt.

11. The method of claim 8, wherein the alkaline aqueous solution comprises at least one of LiOH, NaOH, KOH, and Ca(OH)₂.

12. The method of claim 8, wherein a molar ratio of the silver nanospheres to the phosphine ligand is in a range of 2:1 to 1:2.

13. The method of claim 8, wherein the laser light source is obtained from a monochromatic LED light having a wavelength of from 400 to 500 nm under a powder of from 120 to 180 watts (W).

14. The method of claim 13, wherein the monochromatic LED light has a wavelength of about 455 nm under a powder of about 150 W.

15. A method of obtaining a Raman spectrum of an analyte in a solution, the method comprising:
mixing the solution with the SERS system of claim 1 to form a sample;
exposing the sample to Raman laser light such that a portion of the Raman laser light is scattered by the sample to form scattered light; and
detecting the scattered light;
wherein the analyte is N-acetyl procainamide (NAPA).

16. The method of claim 15, wherein the solution is human blood.

17. The method of claim 15, wherein the scattered light is monitored from 400-2,000 cm⁻¹.

18. The method of claim 15, further comprising quantifying the amount of NAPA present in the solution based on the intensity of the scattered light.

19. The method of claim 18, wherein the intensity of the scattered light linearly correlates with the amount of NAPA present in the solution.

20. The method of claim 18, wherein a linear dynamic range of NAPA present in the solution is from 0.5×10⁻¹² to 0.5×10⁻⁴ M.
